(19) United States
(12) Patent Application Publication
(10) Pub. No.: US 2021/0145275 A1
(43) Pub. Date: May 20, 2021

(54) PUPIL ESTIMATION DEVICE AND PUPIL ESTIMATION METHOD

(71) Applicant: DENSO CORPORATION, Kariya-city (JP)

(72) Inventor: Kaname OGAWA, Kariya-city (JP)

(21) Appl. No.: 17/161,043

(22) Filed: Jan. 28, 2021

Related U.S. Application Data
(63) Continuation of application No. PCT/JP2019/029828, filed on Jul. 30, 2019.

(30) Foreign Application Priority Data
Jul. 31, 2018 (JP) 2018-143754

(51) Int. Cl. A61B 3/113; G06K 9/62

(52) U.S. Cl. CPC A61B 3/113; G06K 9/6215; G06K 9/6257

(57) ABSTRACT

A pupil estimation device is provided as follows. A reference position is calculated using detected peripheral points of an eye in a captured image. A difference vector representing a difference between a pupil central position and the reference position is calculated with a regression function by using (i) the reference position and (ii) a brightness of a predetermined region in the captured image. The pupil central position is obtained by adding the calculated difference vector to the reference position.

### CROSS REFERENCE TO RELATED APPLICATIONS

The present application is a continuation application of International Patent Application No. PCT/JP2019/029828 filed on Jul. 30, 2019, which designated the U.S. and claims the benefit of priority from Japanese Patent Application No. 2018-143754 filed on Jul. 31, 2018. The entire disclosures of all of the above applications are incorporated herein by reference.

### TECHNICAL FIELD

The present disclosure relates to a technique for estimating the central position of a pupil from a captured image.

### BACKGROUND

A method for detecting a specific object contained in an image has been studied. There is disclosed a method for detecting a specific object contained in an image by using machine learning. There is also disclosed a method for detecting a specific object contained in an image by using a random forest or a boosted tree structure.

### SUMMARY

According to an example of the present disclosure, a pupil estimation device is provided as follows. A reference position is calculated using detected peripheral points of an eye in a captured image. A difference vector representing a difference between a pupil central position and the reference position is calculated with a regression function by using (i) the reference position and (ii) a brightness of a predetermined region in the captured image. The pupil central position is obtained by adding the calculated difference vector to the reference position.

### BRIEF DESCRIPTION OF DRAWINGS

The objects, features, and advantages of the present disclosure will become more apparent from the following detailed description made with reference to the accompanying drawings. In the drawings:

FIG. 1 is a block diagram showing a configuration of a pupil position estimation system;

FIG. 2 is a diagram illustrating a method of estimating a central position of a pupil;

FIG. 3 is a diagram illustrating a regression tree according to an embodiment;

FIG. 4 is a diagram illustrating a method of setting the position of a pixel pair using a similarity matrix;

FIG. 5 is a flowchart of a learning process; and

FIG. 6 is a flowchart of a detection process.

### DETAILED DESCRIPTION

Hereinafter, embodiments of the present disclosure will be described with reference to the drawings.

### 1. First Embodiment

[1-1. Configuration]

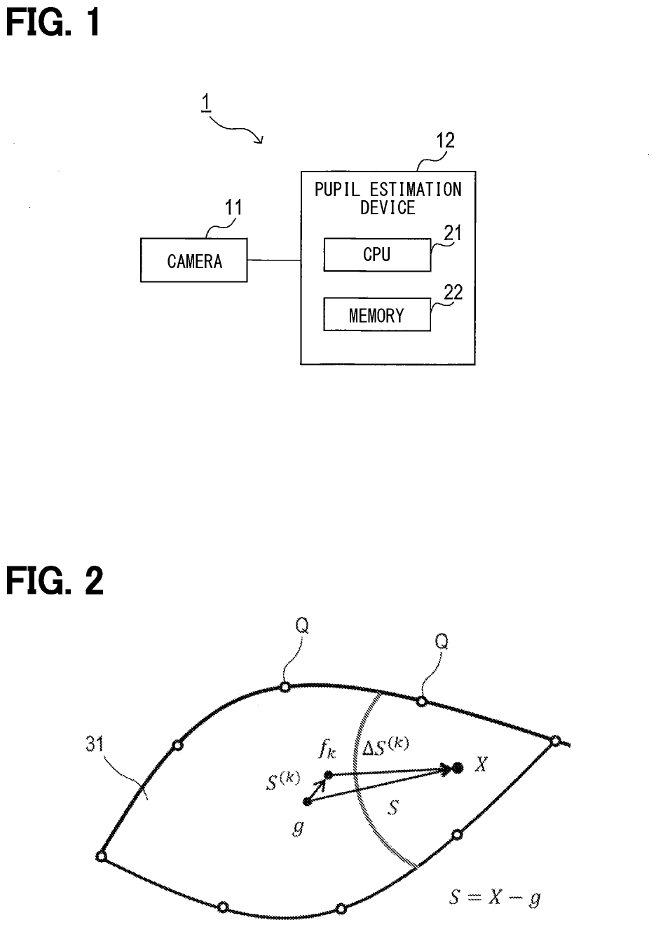

A pupil position estimation system 1 shown in FIG. 1 is a system including a camera 11 and a pupil estimation device 12.

The camera 11 includes a known CCD image sensor, or CMOS image sensor. The camera 11 outputs the captured image data to the pupil estimation device 12.

The pupil estimation device 12, which may also referred to as an information processing device, includes a microcomputer having a CPU 21, and a semiconductor memory such as RAM, ROM (hereinafter, memory 22). Each function of the pupil estimation device 12 is realized by the CPU 21 executing a program stored in a non-transitory tangible storage medium. In this example, the memory 22 corresponds to a non-transitory tangible storage medium storing a program. Further, by execution of this program, a method corresponding to the program is executed. The pupil estimation device 12 may include one microcomputer or a plurality of microcomputers.

[1-2. Estimation Method]

A method of estimating a pupil central position from a captured image including an eye will be described. The pupil central position is the central position of the pupil of the eye. More specifically, it is the center of the circular region that constitutes the pupil. The pupil estimation device 12 estimates the pupil central position by the method described below.

As shown in FIG. 2, the estimated position of the center of the pupil can be obtained by using the following Expression (1).

(Expression (1))

X=g+S (1)

X: Estimated position vector of the center of the pupil

g: Center of gravity position vector determined from the peripheral points around the eye

S: Difference vector between the estimated pupil central position and the center of gravity position

The method of estimating the center of gravity position vector g and the difference vector S will be described below.

(i) Calculation of Center of Gravity Position Vector g

A method of estimating the center of gravity position vector g will be described with reference to FIG. 2. The center of gravity position is the position of the center of gravity of the eye region 31, which is the region where the eyeball is displayed in the captured image. The center of gravity position vector g is obtained based on a plurality of peripheral points Q around an eye; the peripheral points are feature points indicating the outer edge portion of the eye region 31. The method for obtaining the peripheral points Q is not particularly limited, and the peripheral points Q can be obtained by various methods capable of obtaining the center of gravity position vector g. For example, it can be obtained by the feature point detection as disclosed in Reference 1 or the method using Active Shape Model.

Reference 1: One Millisecond Face Alignment with an Ensemble of Regression Trees (Vahid Kazemi and Josephine Sullivan, The IEEE Conference on CVPR, 2014, 1867-1874), which is incorporated herein by reference.

Note that FIG. 2 illustrates the eight peripheral points Q including (i) two corner points of the outer corner and the inner corner of the eye, and (ii) six intersections between the outer edge of the eye region 31 and three vertical straight lines cutting the straight line connecting the two corner points into quarters. The number of peripheral points Q is not limited to this. The position of the center of gravity is, for example, the average position of a plurality of peripheral points Q of the eye. The positions of the peripheral points Q are appropriately dispersed at the outer edge of the eye region 31, so that the accuracy of the center of gravity position vector g is improved.

(Ii) Calculation of Difference Vector S

The difference vector S can be represented by the function shown in the following Expression (2).

(Expression (2))

S=f_{K}(S^{(K)}) (2)

Further, f_{K}(S^{(K)}) of Expression (2) can be expressed by the function shown in the following Expression (3).

$$\begin{matrix}( Expression ( 3 ) ) & \\ {f}_{K} ( {S}^{( K )} ) = {f}_{0} ( {S}^{( 0 )} ) + v {\sum }_{k = 1}^{K} {g}_{k} ( {S}^{( k )} ) & ( 3 )\end{matrix}$$

In Expression (3), gₖ is a regression function. Further, K is the number of additions of the regression function, that is, the number of iterations. Practical accuracy can be obtained by setting K to several tens of times or more, for example.

As shown in the above Expressions (2) and (3), the pupil estimation method of the present embodiment applies the function f_{K} to the current difference vector S^{(K)}. By doing so (in other words, by making corrections using the regression function gₖ), the updated difference vector S^{(K+1)} is obtained. Then, by repeating this, the difference vector S is obtained as a final difference vector with improved accuracy.

Here, f_{K} is a function, which includes a regression function gₖ, and applies the additive model of the regression function using Gradient Boosting. The additive model is indicated by the above-mentioned Reference 1 or following Reference 2.

Reference 2: Greedy Function Approximation: A gradient boosting machine (Jerome H. Friedman, The Annals of Statistics Volume 29, Number 5 (2001), 1189-1232), which is incorporated herein by reference.

Hereinafter, each element of Expression (3) will be described.

(ii-1) Initial Value f₀(S⁽⁰⁾)

In the above Expression (3), the initial value fₒ(S⁽⁰⁾) is obtained as shown in the following Expressions (4) and (5) based on a plurality of images used as learning samples.

$$\begin{matrix}( Expression ( 4 ) ) & \\ {f}_{0} ( {S}^{( 0 )} ) = {arg min}_{γ} {\sum }_{i = 1}^{N} {‖ Δ {S}_{i}^{( 0 )} - γ ‖}^{2} & ( 4 ) \\ ( Expression ( 5 ) ) & \\ Δ {S}_{i}^{( 0 )} = {S}_{{π}_{i}} - {S}_{i}^{( 0 )} & ( 5 )\end{matrix}$$

Here, the parameters are as follows.

N: Number of images in the training sample

i: Index of the learning sample

S_{π}: Teacher data showing the correct pupil central position of the training sample

v: Parameter that controls the effectiveness of regression learning, 0<v<1

S⁽⁰⁾: Average pupil position of multiple training samples

The above-mentioned f₀(S⁽⁰⁾) is a value when γ is input so that the right side is the smallest in Expression (4).

(ii-2) Regression Function gₖ(S^{(k)})

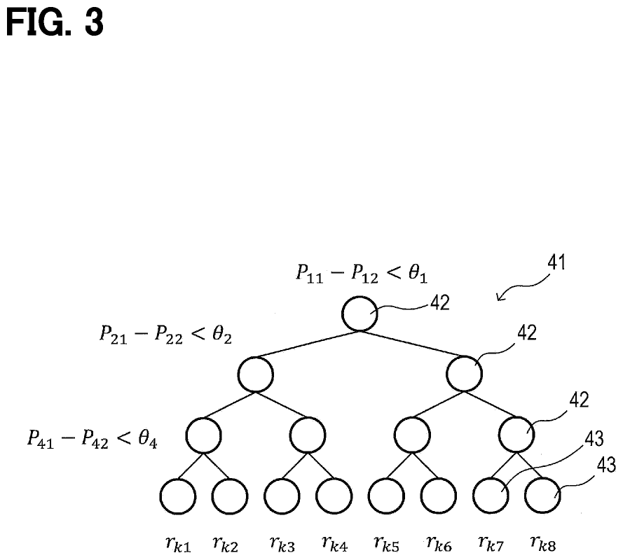

The regression function gₖ(S^{(k)}) in above Expression (3) is a regression function that takes the current difference vector S^{(k)} as a parameter. The regression function gₖ(S^{(k)}) is obtained based on the regression tree 41 as shown in FIG. 3, as described in reference 2. The regression function gₖ(S^{(k)}) is a relative displacement vector representing the moving direction and the moving amount in the captured image plane. This regression function gₖ(S^{(k)}) corresponds to a correction vector used to correct the difference vector S.

In each node 42 of the regression tree 41, the brightness difference between the combination of two pixels (hereinafter referred to as a pixel pair) defined by the relative coordinates from the current pupil prediction position (g+S^{(k)}) is compared with the predetermined threshold θ. Then, the left-right direction to be followed by the regression tree 41 is determined according to whether the brightness difference is higher or lower than the threshold. A regression amount rk is defined for each leaf 43 (i.e., each end point) of the regression tree 41. This regression amount rk is the value of the regression function gₖ(S^{(k)}) with respect to the current pupil prediction position (g+S^{(k)}). The position obtained by adding the current different vector S^{(k)} to the position of the center of gravity g corresponds to the current pupil prediction position (g+S^{(k)}) as a temporary pupil central position. The regression tree 41 (i.e., the pixel pair and threshold of each node, and the regression amount rk set at the end point (that is, the leaf 43 of the regression tree 41)) is obtained by learning. As the position of the pixel pair, a value corrected as described later is used.

The reason for using the brightness difference of the pixel pair as the input information is as follows. Each node 42 of the regression tree 41 determines whether one of the two pixels constitutes a pupil portion and the other constitutes a portion other than the pupil. In the captured image, the pupil portion is relatively dark in color, and the portion other than the pupil is relatively light in color. Therefore, by using the brightness difference of the pixel pair as the input information, the above-mentioned determination can be easily performed.

Using the regression function gₖ(S^{(k)}) obtained in this way, the difference vector S^{(k)} can be updated by the following Expression (6).

(Expression (6))

S^{(k+1)}=fₖ(S^{(k)})+vgₖ(S^{(k)}) (6)

By reducing the value of v, overfitting is suppressed to respond to the diversity of pupil central positions. Note that fₖ(S^{(k)}) in the Expression (6) is the difference vector that has undergone the k−1th update, and vgₖ(S^{(k)}) is the correction amount in the kth update.

(ii-2-1) Pixel Pair Position

The position of the pixel pair is determined for each node 42 in the regression tree 41 for obtaining the regression function gₖ(S^{(k)}). The pixel position in the captured image of each pixel pair referred to in the regression tree 41 is a coordinate position determined by relative coordinates from the temporary pupil central position (g+S^{(k)}) at that time. Here, the vector that determines the relative coordinates is a modified vector (i.e., modified standard vector) that is obtained by adding a modification using a similarity matrix to a standard vector predetermined to a standard image. The similarity matrix (hereinafter, transformation matrix R) is to reduce the amount of deviation between the eye in the standard image and the eye in the captured image. The standard image referred to here is an average image obtained from a large number of training samples.

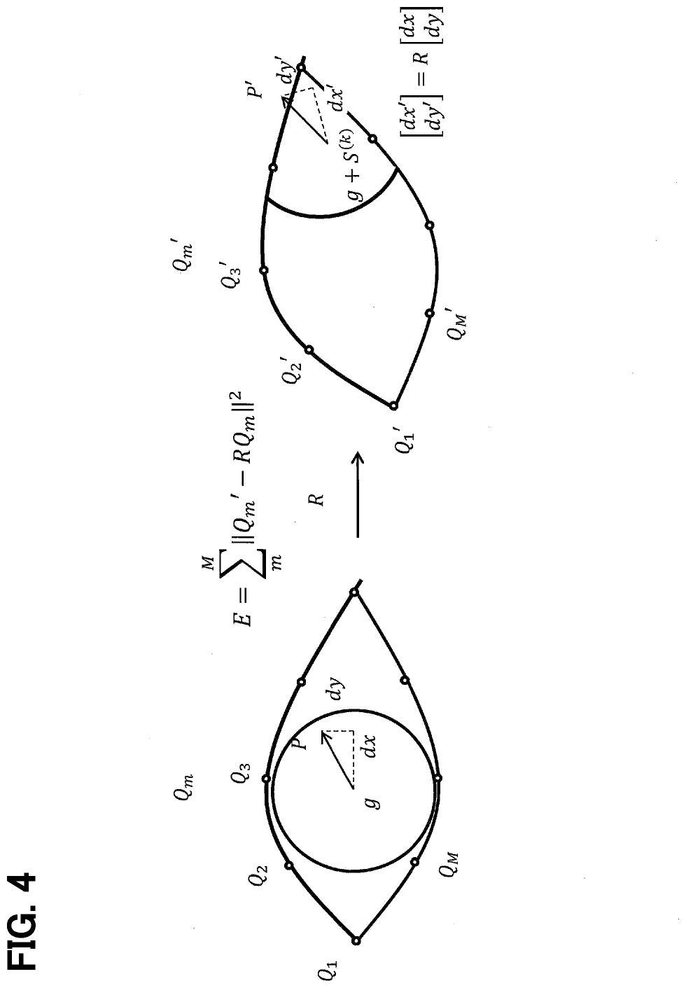

A method of specifying the position of the pixel pair will be specifically described with reference to FIG. 4. The figure on the left side of FIG. 4 is a standard image, and the figure on the right side is a captured image. The standard vector predetermined for the standard image is (dx, dy). The modified vector that is obtained by adding a modification using the similarity matrix to the standard vector is (dx′, dy′).

In advance, M eye peripheral points Q for a plurality of learning samples are obtained, and M Qm are learned as the average position of each point. Then, M Qm′ are calculated from the captured image in the same manner as the peripheral points from the standard image. Then, the transformation matrix R that minimizes the following Expression (7) is obtained between Qm and Qm′. Using this transformation matrix R, the position of the pixel pair relatively determined at a certain temporary pupil central position (g+S^{(k)}) is set by the following Expression (8).

$$\begin{matrix}( Expression ( 7 ) ) & \\ E = {\sum }_{m}^{M} {‖ {Q}_{{m}^{′}} - R {Q}_{m} ‖}^{2} & ( 7 ) \\ ( Expression ( 8 ) ) & \\ [ \begin{matrix}{P}_{ϰ} \\ {P}_{y}\end{matrix} ] = [ \begin{matrix}{g}_{ϰ} \\ {g}_{y}\end{matrix} ] + [ \begin{matrix}{S}_{ϰ}^{( k )} \\ {S}_{y}^{( k )}\end{matrix} ] + R [ \begin{matrix}d x \\ dy\end{matrix} ] & ( 8 )\end{matrix}$$

The transformation matrix R is a matrix showing what kind of rotation, enlargement, and reduction should be applied to the average value Qm based on a plurality of training samples to be the closest to the Qm′ of the target training sample. By using this transformation matrix R, the position of the pixel pair can be set by using the modified vector in which the deviation between the standard image and the captured image is offset as compared with the standard vector. Although it is not essential to use this transformation matrix R, it is possible to improve the detection accuracy of the center of the pupil by using the transformation matrix R.

(iii) Outline

As described above, in the present embodiment, the regression function estimation for obtaining the difference vector S is performed using the brightness difference of the pixel pair of two different points set in each node 42 of the regression tree 41. Further, in order to determine the regression tree 41 (regression function gk), Gradient Boosting is performed to obtain the relationship between the brightness difference and the pupil position. The information input to the regression tree 41 does not have to be the brightness difference of the pixel pair. For example, the absolute value of the brightness of the pixel pair may be used, or the average value of the brightness in a certain range may be obtained. That is, various information regarding the brightness around the temporary pupil central position can be used as input information. However, it is convenient to use the brightness difference of the pixel pair because the feature amount thereof tends to be large, and it is possible to suppress an increase in the processing load.

[1-3. Process]

The pupil estimation device 12 obtains the regression tree 41, the pixel pair based on the average image, and the threshold 8 by performing learning in advance. Further, the pupil estimation device 12 efficiently estimates the pupil position from the detection target image, which is a captured image obtained by the camera 11, by using the regression tree 41, the pixel pair, and the threshold 8 obtained by learning. It should be noted that the learning in advance does not necessarily have to be performed by the pupil estimation device 12. The pupil estimation device 12 can use information such as a regression tree obtained by learning by another device.

[1-3-1. Learning Process]

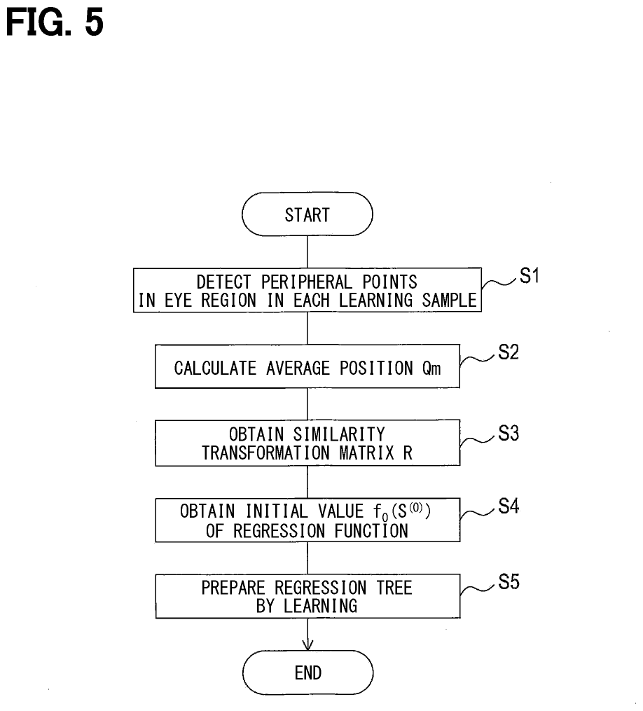

The learning process executed by the CPU 21 of the pupil estimation device 12 will be described with reference to the flowchart of FIG. 5.

First, in S1, the CPU 21 detects the peripheral points Q of the eye region for each of a plurality of learning samples.

In S2, the CPU 21 calculates the average position Qm of the peripheral points Q for each of all the learning samples.

In S3, the CPU 21 obtains a Similarity transformation matrix R for each learning sample. As described above, this Similarity transformation matrix R is a transformation matrix that minimizes Expression (7), as described above.

In S4, the CPU 21 obtains the initial value f₀(S⁽⁰⁾) of the regression function by using Expression (4).

In S5, the CPU 21 configures the regression tree used for estimating the pupil center (i.e., the position and threshold of the pixel pair with respect to each node) by learning using so-called gradient boosting. Here, first, (a) the regression function gₖ implemented as a regression tree is obtained. The method of dividing each binary tree at this time may employ the method described in Section 2.3.2 of Reference 1 “One Millisecond Face Alignment with an Ensemble of Regression Trees” described above, for instance. Then, (b) the regression tree is applied to each learning sample, and the current pupil position is updated using above-mentioned Expression (3). After the update in (b), the above (a) is performed again to obtain the regression function gk, and then the above (b) is performed. This is repeated K times, and the regression tree is configured by learning.

After this S5, this learning process is completed.

[1-3-2. Detection Process]

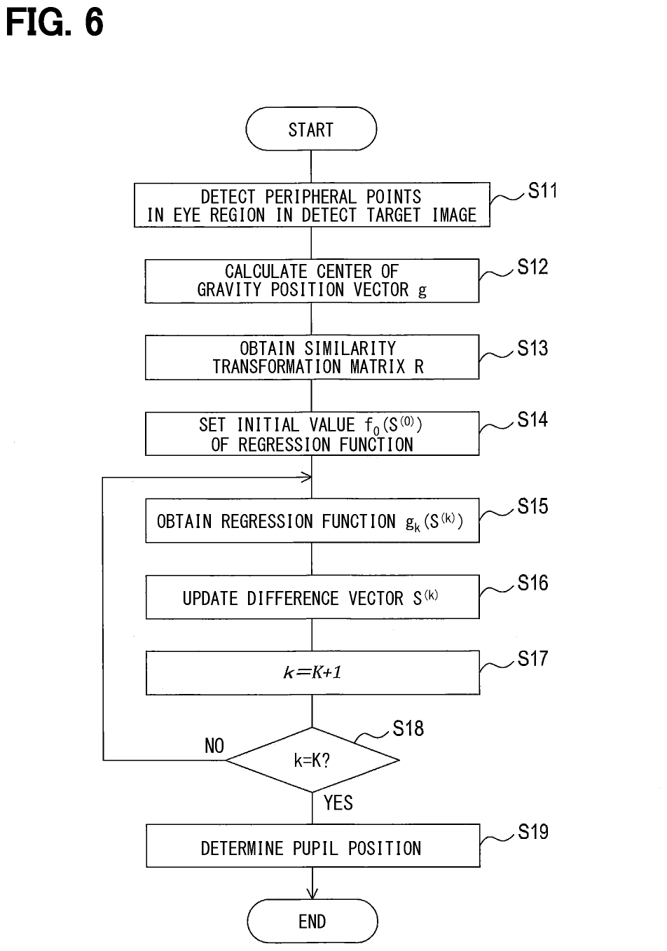

Next, the detection process executed by the CPU 21 of the pupil estimation device 12 will be described with reference to the flowchart of FIG. 6.

First, in S11, the CPU 21 detects the peripheral points Q in the eye region 31 in the detection target image. This S11 corresponds to the processing of a peripheral point detection unit.

In S12, the CPU 21 calculates the center of gravity position vector g from the peripheral points Q obtained in S11. This S12 corresponds to the processing of a position calculation unit.

In S13, the CPU 21 obtains the Similarity transformation matrix R for the image for the detection target image. The pixel position of the pixel pairs used in each node 42 of the regression tree 41 is determined by learning in advance, but it is only a relative position based on the above-mentioned standard image. Therefore, the target pixel position is modified in the detection target image by using the Similarity transformation matrix R that approximates the standard image to the detection target image. As a result, the pixel position becomes more suitable for the regression tree generated by learning, and the detection accuracy of the center of the pupil is improved. The Qm used in Expression (7) may employ the value obtained by learning in S2 of FIG. 5. This S13 corresponds to the processing of a matrix obtainment unit.

In S14, the CPU 21 initializes with k=0. Note that f₀(S⁽⁰⁾) may employ the value obtained by learning in S4 of FIG. 5.

In S15, the CPU 21 obtains the regression function gₖ(S^{(k)}) by tracing the learned regression tree. This S15 corresponds to the processing of the correction amount calculation unit.

In S16, the CPU 21 uses the gₖ(S^{(k)}) obtained in S15 and adds gₖ(S^{(k)}) to S^{(k)} based on the above Expression (6). By doing so, the difference vector S^{(k)} for specifying the current pupil position is updated. This S16 corresponds to the processing of the update unit. Further, in the following S17, k=k+1.

In S18, the CPU 21 determines whether or not k=K. This K can be, for example, a value of about several tens. If k=K, that is, if the update by S15 and S16 is repeated a predetermined number of times, the process shifts to S19. On the other hand, if k is not equal to K, that is, if the update by S15 and S16 is not repeated K times, the process returns to S15. This S18 corresponds to the processing of a computation control unit. Further, the processing of S13 to S18 corresponds to the processing of a first computation unit.

In S19, the CPU 21 determines the pupil position on the detection target image according to Expression (1) by using the difference vector S^{(K)} (i.e., finally obtained difference vector or final difference vector) obtained in the last S17 and the center of gravity position vector g obtained in S12. That is, in S19, the estimated value of a final pupil central position (i.e., finally updated pupil central position) is calculated. After that, this detection process ends. This S19 corresponds to the processing of a second computation unit.

[1-4. Effects]

According to the embodiment described in detail above, the following effects are obtained.

(1a) In the present embodiment, the difference vector between the position of the center of gravity and the position of the pupil is functionally predicted by using the method of the regression function, thereby estimating the position of the center of the pupil. Therefore, for example, the position of the center of the pupil (i.e., pupil central position) can be estimated efficiently as compared with the method of specifying the position of the pupil by repeatedly executing the sliding window.

(1b) In the present embodiment, the brightness difference of a predetermined pixel pair is used as the input information to the regression tree. Therefore, it is possible to obtain a suitable value in which the feature amount tends to be large with a low load as compared with the case using as input information other information such as an absolute value of brightness or a brightness in a certain range.

(1b) In the present embodiment, a similarity matrix is used to convert a standard vector into a modified vector (i.e., modified standard vector) to specify a pixel pair and obtain a brightness difference. Therefore, it is possible to estimate the pupil central position with high accuracy by reducing the influence of the size and angle of the eye on the detection target image.

### 2. Other Embodiments

Although the embodiment of the present disclosure has been described above, the present disclosure is not limited to the above-described embodiment, and it is possible to implement various modifications.

(3a) In the above embodiment, a configuration in which the center of gravity position vector g is calculated using a plurality of peripheral points Q is described. However, the reference position calculated using the peripheral points Q is not limited to the position of the center of gravity. In other words, the reference position of the eye is not limited to the position of the center of gravity, and various positions can be used as a reference or a reference position. For example, the midpoint between the outer and inner corners of the eye may be used as a reference position.

(3b) In the above embodiment, a method of obtaining a regression function gₖ(S^{(k)}) using a regression tree is described. However, if the method uses a regression function, it is not necessary to use a regression tree. Moreover, although the method of configuring the regression tree by learning using Gradient Boosting is described, the regression tree may be configured by another method.

(3c) In the above embodiment, a configuration in which the difference vector S^{(k)} is updated a plurality of times to obtain the pupil center is described. However, there is no need to be limited to this. The pupil center may be obtained by adding the difference vector only once. Further, the number of times the difference vector is updated, in other words, the condition for ending the update is not limited to the above embodiment, and may be configured to repeat until some preset condition is satisfied.

(3d) In the above embodiment, the configuration in which the position of the pixel pair for calculating the brightness difference inputted to the regression tree is modified by using the Similarity matrix is described. However, the configuration may not use the Similarity matrix.

(3e) A plurality of functions of one element in the above embodiment may be implemented by a plurality of elements, or one function of one element may be implemented by a plurality of elements. Further, a plurality of functions of a plurality of elements may be implemented by one element, or one function implemented by a plurality of elements may be implemented by one element. In addition, a part of the configuration of the above embodiment may be omitted. At least a part of the configuration of the above embodiment may be added to or substituted for the configuration of the other above embodiment.

(3f) The present disclosure can be also realized, in addition to the above-mentioned pupil estimation device 12, in various forms such as: a system including the pupil estimation device 12 as a component, a program for operating a computer as the pupil estimation device 12, a non-transitory tangible storage medium such as a semiconductor memory in which this program is stored, and a pupil estimation method.

For reference to further explain features of the present disclosure, the description is added as follows.

A method for detecting a specific object contained in an image has been studied. There is disclosed a method for detecting a specific object contained in an image by using machine learning. There is also disclosed a method for detecting a specific object contained in an image by using a random forest or a boosted tree structure.

However, detailed examination by the inventor has found that the above methods are not efficient and it is difficult to detect a pupil at high speed with high accuracy. This is, the methods in the above each use a detection unit that have been trained to respond to a specific pattern in a window. This detection unit moves to change the position and/or size on the image by the method of the sliding window and discovers matching patterns while scanning sequentially. In such a configuration, windows, which are cut out at different sizes and positions, need to be evaluated many times. Also, most of the windows that should be evaluated each time may overlap with the previous one. It is thus inefficient and there is much room for improvement in terms of speed and memory bandwidth. Also, in the sliding window method, if there are variations in the angle of the object to be detected, it is necessary to configure the detection unit for each angle range to some extent. In this respect as well, the efficiency may be not good.

It is thus desired to provide a technique capable of efficiently estimating the central position of a pupil.

Aspects of the present disclosure described herein are set forth in the following clauses.

According to a first aspect of the present disclosure, a pupil estimation device is provided to include a peripheral point detection unit, a position calculation unit, a first computation unit, and a second computation unit. The peripheral point detection unit is configured to detect a plurality of peripheral points each indicating an outer edge of an eye, from the captured image. The position calculation unit is configured to calculate a reference position using the plurality of peripheral points detected by the peripheral point detection unit. The first computation unit is configured to calculate a difference vector representing a difference between the pupil central position and the reference position with a regression function by using (i) the reference position calculated by the position calculation unit and (ii) a brightness of a predetermined region in the captured image. The second computation unit is configured to calculate the pupil central position by adding the difference vector calculated by the first computation unit to the reference position.

According to a second aspect of the present disclosure, a pupil estimation method is provided as follows. In the method, a plurality of peripheral points each indicating an outer edge of an eye are detected from a captured image. A reference position is calculated using the plurality of peripheral points. Using the reference position and a brightness of a predetermined region in the captured image, a difference vector representing a difference between the pupil central position and the reference position is calculated with a regression function. The pupil central position is calculated by adding the calculated difference vector to the reference position.

The above configurations of both the aspects can estimate efficiently the pupil central position by using the regression function, while suppressing the decrease in efficiency due to the use of the sliding window.

What is claimed is:

1. A pupil estimation device that estimates a pupil central position from a captured image, comprising:
a peripheral point detection unit configured to detect a plurality of peripheral points each indicating an outer edge of an eye, from the captured image;
a position calculation unit configured to calculate a reference position using the plurality of peripheral points detected by the peripheral point detection unit;
a first computation unit configured to calculate a difference vector representing a difference between the pupil central position and the reference position with a regression function by using (i) the reference position calculated by the position calculation unit and (ii) a brightness of a predetermined region in the captured image; and
a second computation unit configured to calculate the pupil central position by adding the difference vector calculated by the first computation unit to the reference position,
wherein
the first computation unit comprises:
a correction amount calculation unit configured to perform a correction-vector calculation that calculates a correction vector representing a movement direction and a movement amount in the captured image to correct the difference vector, wherein the pupil central position calculated by adding the difference vector to the reference position is defined as a temporary pupil central position, and brightness information around the temporary pupil central position is used as input information;
an update unit configured to perform a difference-vector update that updates the difference vector by adding the correction vector calculated by the correction amount calculation unit to the difference vector; and
a computation control unit configured to repeatedly perform, until a preset condition is satisfied, a sequence of (i) the correction-vector calculation by the correction amount calculation unit using the difference vector updated by the update unit, and (ii) the difference-vector update by the update unit using the correction vector calculated by the correction amount calculation unit,
wherein
(i) the correction amount calculation unit is configured to perform the correction-vector calculation by using a regression tree,
(ii) in the regression tree, the correction vector is set at each end point, and a brightness difference between two pixels as a pixel pair set with reference to the temporary pupil central position is used as input information at each node, and
(iii) the regression tree is configured using Gradient Boosting,
the pupil estimation device further comprising:
a matrix obtainment unit configured to obtain a similarity matrix that reduces an amount of deviation between the plurality of peripheral points of the eye in the captured image and a plurality of peripheral points of an eye in a standard image,
wherein
a position of the pixel pair is obtained by modifying a standard vector predetermined to the standard image using the similarity matrix obtained by the matrix obtainment unit, and adding the modified standard vector to the temporary pupil central position.

2. The pupil estimation device according to claim 1, wherein:
the reference position is a position of a center of gravity of the eye.

3. A computer-implemented pupil estimation method executed by a computer, comprising:
(a) calculating a reference position using a plurality of peripheral points of an eye, which are detected from a captured image;
(b) obtaining a similarity matrix that reduces an amount of deviation between the plurality of peripheral points of the eye in the captured image and a plurality of peripheral points of an eye in a standard image;
(c) performing a correction-vector calculation that calculates a correction vector in the captured image as a regression function by tracing a regression tree, to correct a difference vector that indicates a difference between the reference position and a temporary pupil central position,
wherein
(i) at each end point in the regression tree, the correction vector is set,
(ii) at each node in the regression tree, a brightness difference between two pixels of a pixel pair set with reference to the temporary pupil central position is used as input information,
(iii) a position of the pixel pair is obtained by modifying a standard vector predetermined to the standard image using the similarity matrix, and adding the modified standard vector to the temporary pupil central position, and
(iv) the regression tree is configured using Gradient Boosting;
(d) performing a difference-vector update after performing the correction-vector calculation that calculates the correction vector, the difference-vector update adding the calculated correction vector to the difference vector to update the difference vector;
(e) performing repeatedly, until a preset condition is satisfied, a sequence of (i) the correction-vector calculation using the updated difference vector to provide the calculated correction vector and (ii) the difference-vector update using the calculated correction vector, to finally update the difference vector; and
(f) calculating a pupil central position by adding the finally undated difference vector to the reference position.

4. A pupil estimation device, comprising:
one or more processors coupled with one or more memories and a camera via a communication link, the one or more processors configured to:
(a) calculate a reference position using a plurality of peripheral points of an eye, which are detected from a captured image by the camera;
(b) obtain a similarity matrix that reduces an amount of deviation between the plurality of peripheral points of the eye in the captured image and a plurality of peripheral points of an eye in a standard image;
(c) perform a correction-vector calculation that calculates a correction vector in the captured image as a regression function by tracing a regression tree, to correct a difference vector that indicates a difference between the reference position and a temporary pupil central position,
wherein
(i) at each end point in the regression tree, the correction vector is set,
(ii) at each node in the regression tree, a brightness difference between two pixels of a pixel pair set with reference to the temporary pupil central position is used as input information,
(iii) a position of the pixel pair is obtained by modifying a standard vector predetermined to the standard image using the similarity matrix, and adding the modified standard vector to the temporary pupil central position, and
(iv) the regression tree is configured using Gradient Boosting;
(d) perform a difference-vector update after performing the correction-vector calculation that calculates the correction vector, the difference-vector update adding the calculated correction vector to the difference vector to update the difference vector;
(e) perform repeatedly, until a preset condition is satisfied, a sequence of (i) the correction-vector calculation using the updated difference vector to provide the calculated correction vector and (ii) the difference-vector update using the calculated correction vector, to finally update the difference vector; and
(f) calculate a pupil central position by adding the finally undated difference vector to the reference position.
